(19) United States
(12) Patent Application Publication
(10) Pub. No.: US 2022/0356164 A1
(43) Pub. Date: Nov. 10, 2022

(54) 2-AMINOSELENOPHEN COMPOUNDS WITH ANTI-DRUG RESISTANT BACTERIA ACTIVITY AND A METHOD OF PREPARING THE SAME

(71) Applicants: Wenqiang CAO, Zhuhai (CN); Chang LIU, Zhuhai (CN); Maoxun YANG, Zhuhai (CN); Haiwei LI, Zhuhai (CN); Yikang CHEN, Zhuhai (CN)

(72) Inventors: Wenqiang CAO, Zhuhai (CN); Chang LIU, Zhuhai (CN); Maoxun YANG, Zhuhai (CN); Haiwei LI, Zhuhai (CN); Yikang CHEN, Zhuhai (CN)

(73) Assignee: ZHUHAI JINAN SELENIUM SOURCE NANOTECHNOLOGY CO., LTD., Zhuhai (CN)

(21) Appl. No.: 17/378,696

(22) Filed: Jul. 17, 2021

(30) Foreign Application Priority Data
Apr. 22, 2021 (CN) 202110438493.7

(51) Int. Cl. C07D 345/00

(52) U.S. Cl. CPC C07D 345/00

(57) ABSTRACT

A compound with anti-drug resistant bacteria activity having the following formula (I):

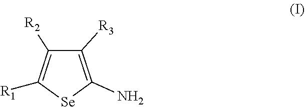

is disclosed. R₁ and R₂ are each independently H, alkyl, or phenyl, or R₁ and R₂ form —(CH₂)₃—, —(CH₂)₄—, or —(CH₂)₅—, and R₃ is —COOCH₂CH₃, or CN. A method of preparing the compound of formula (I) is also disclosed.

This application claims priority to Chinese Patent Application No. 202110438493.7, filed on Apr. 22, 2021, which is incorporated by reference for all purposes as if fully set forth herein.

### FIELD OF THE INVENTION

The present invention relates to the field of medicinal chemistry, and in particular, to 2-aminoselenophen compounds with anti-drug resistant bacteria activity and a method of preparing the same.

### BACKGROUND OF THE INVENTION

Selenium-containing heterocyclic compounds, such as selenophene compounds, have a variety of biological and pharmaceutical activities, and have good photoelectric properties due to the unique structure of selenium atoms, so they are widely used in the synthesis of new organic photoelectric materials and drugs. The preparation of selenophene was reported more than 80 years ago, but effective preparation methods are still lack. The preparation of selenophenes reported so far uses the reaction of unsaturated alkenyne with the corresponding selenium, and the reaction conditions are harsh. In 1927, Mazza et al. reported for the first time that selenium and acetylene reacted at a high temperature of 400° C., and selenophene was detected in the product; in 1928, Briscoe et al. also reported that selenophene was prepared by the same method with a yield of 15%; in 1954, Yurev et al. heated selenium dioxide and butane (or butene, or butadiene) to 450-500° C. to prepare selenophene in the presence of chromium oxide (or aluminum oxide), and the yield was only 3-13%. These preparation methods have harsh reaction conditions and low yields, and most of the reactions use unstable, difficult-to-prepare, and pungent odor organic selenium compounds as selenium sources, and the reaction operations are relatively cumbersome. Considering the important role of selenophene compounds in new organic photoelectric materials and medical and biological applications, it is necessary to develop convenient and efficient synthetic methods.

The 2-aminoselenophene small molecule compounds are mainly used in the anti-tumor field of the pharmaceutical industry. The present invention introduces a cyano group on the selenophene ring to enhance its interaction with bacterial ribosomal proteins, especially with drug-resistant bacteria ribosomal proteins. The combined effect of the drug makes it have good antibacterial activity and has the potential to become a new type of anti-multidrug resistant bacteria drug.

In the present invention, the method of the present invention uses simple aldehyde or ketone compounds, nitrile compounds and sodium selenide as reaction raw materials, and carries out the synthesis of 2-aminoselenophene compounds under ultrasonic catalysis. Preliminary antibacterial activity experiment indicates that the compound has excellent antibacterial activity and has high medical research and application value in the treatment of infectious diseases caused by multidrug resistant bacteria.

### SUMMARY OF THE INVENTION

In one embodiment, the present invention discloses a compound with anti-drug resistant bacteria activity having the following formula (I):

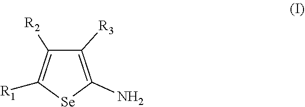

R₁ and R₂ are each independently H, alkyl, or phenyl, or R₁ and R₂ form —(CH₂)₃—, —(CH₂)₄—, or —(CH₂)₅—, and R₃ is —COOCH₂CH₃, or CN.

In another embodiment, the compound is selected from the group consisting of:

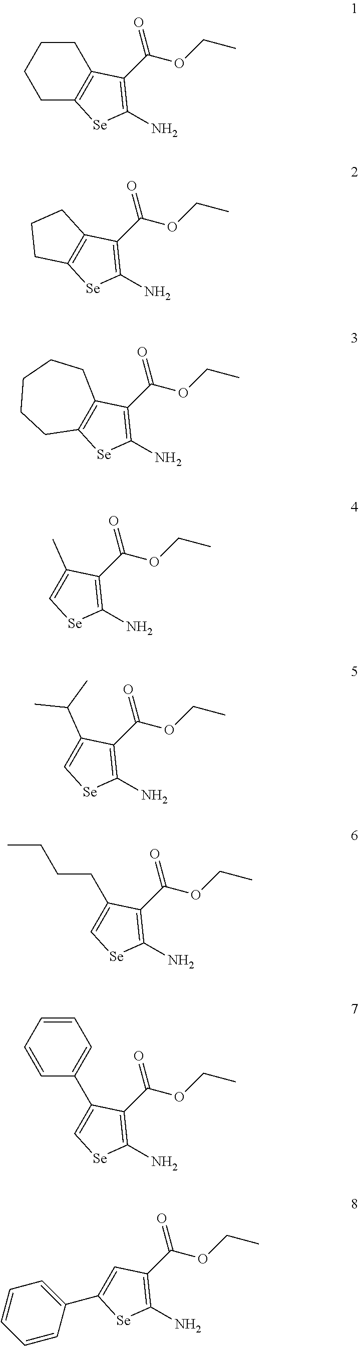

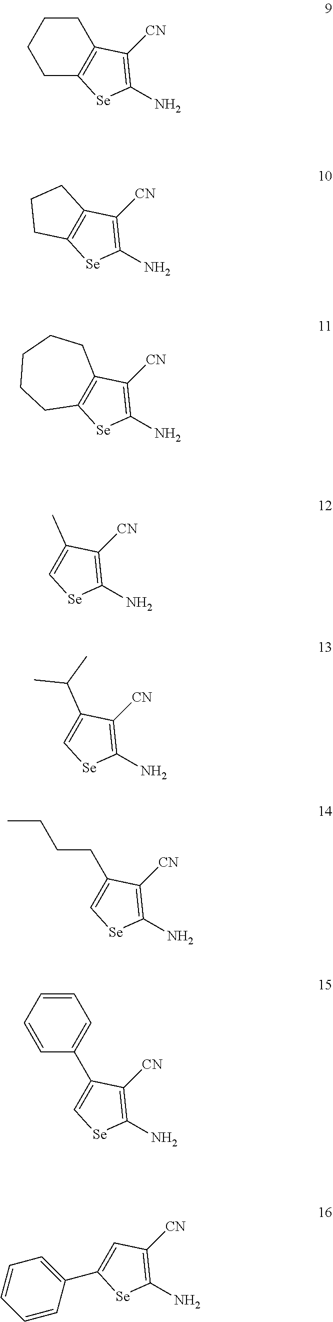

In another embodiment, the present invention discloses a method of preparing the compound of formula (I). The method includes: reacting a compound of formula (II) with a compound of formula (III) and Na₂Se to obtain the compound of formula (I):

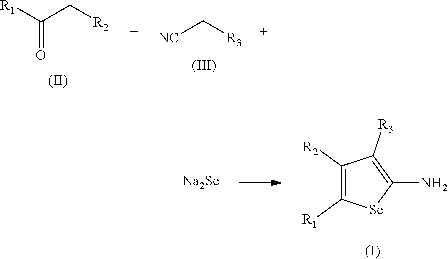

In another embodiment, the method includes the following steps: placing the compound of formula (II), the compound of formula (III) and an organic solvent in a reactor; adding Na₂Se under nitrogen atmosphere to obtain a reaction mixture; heating the reaction mixture at 30-60° C. for 2-5 hours; pouring the reaction mixture system into ice water, standing still for crystallization to obtain a crude product; and recrystallizing the crude product using methanol and drying to obtain the compound of formula (I).

In another embodiment, the organic solvent is methanol or ethanol.

In another embodiment, a molar ratio of the compound of formula (II), the compound of formula (III) and Na₂Se is 1:1: (1.5-2).

In another embodiment, the molar ratio of the compound of formula (II), the compound of formula (III) and Na₂Se is 1:1:1.8.

In another embodiment, the reaction mixture is heated at 45° C.

In another embodiment, the reaction mixture is heated for 3 hours.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 shows the in vitro antibacterial activity of compound 9 against drug-resistant bacteria MARS 18-575.

FIG. 2 shows the in vitro antibacterial activity of compound 9 against drug-resistant bacteria MARS 18-596.

FIG. 3 shows the in vitro antibacterial activity of compound 9 against drug-resistant bacteria MDR-PA 18-756.

FIG. 4 shows the in vitro antibacterial activity of compound 11 against drug-resistant bacteria MARS 18-575.

FIG. 5 shows the in vitro antibacterial activity of compound 11 against drug-resistant bacteria MARS 18-596.

FIG. 6 shows the in vitro antibacterial activity of compound 11 against drug-resistant bacteria MDR-PA 18-126.

FIG. 7 shows the in vitro antibacterial activity of compound 11 against drug-resistant bacteria MDR-PA 18-756.

FIG. 8 shows the in vitro antibacterial activity of compound 14 against drug-resistant bacteria MARS 18-575.

FIG. 9 shows the in vitro antibacterial activity of compound 14 against drug-resistant bacteria MARS 18-596.

FIG. 10 shows the in vitro antibacterial activity of compound 14 against drug-resistant bacteria MDR-PA 18-756.

### DETAILED DESCRIPTION OF THE ILLUSTRATED EMBODIMENTS

Reference will now be made in detail to embodiments of the present invention, example of which is illustrated in the accompanying drawings. The following examples illustrate the present invention, but the present invention is not limited to the following examples.

### Example 1: Preparation of Compound 1

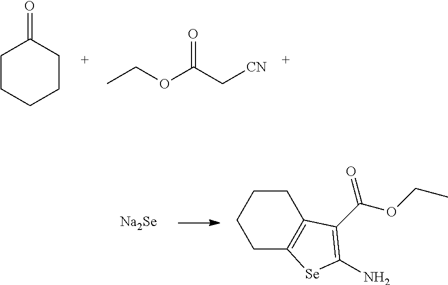

Take 98.0 mg cyclohexanone and 114.0 mg ethyl cyanoacetate (molar ratio 1:1) in a 50 mL single-necked flask, add 20 mL ethanol to dissolve, ultrasonically shake at 45° C., 40 KHz for 20 min, then add 225.0 mg Na₂Se (molar ratio 1:1:1.8), react under nitrogen protection, 45° C., 40 KHz ultrasonic oscillation. At the end of the reaction, the reaction liquid became turbid, the reaction liquid was poured into cold water, left standing, filtered to obtain a crude product, and then further purified by recrystallization with methanol to obtain compound 1 with a mass of 104.8 mg and a yield of 38.5%.

¹H-NMR (400 MHz, DMSO-d₆) δ (ppm): 8.52 (2H, s), 4.25 (2H, m), 2.29 (2H, t), 2.02 (2H, t), 1.73 (2H, m), 1.69 (2H, m), 1.35 (3H, t). ¹³C-NMR (100 MHz, DMSO-d6) δ (ppm): 166.0, 154.6, 145.0, 142.1, 109.0, 63.0, 32.1, 30.2, 28.1, 23.6, 15.8.

### Example 2: Preparation of Compound 2

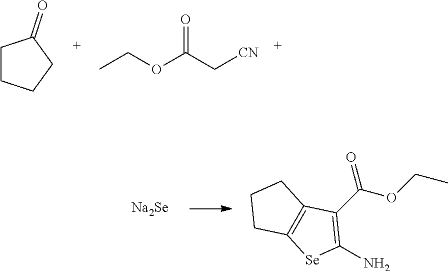

Take 84.0 mg cyclopentanone and 114.0 mg ethyl cyanoacetate (molar ratio 1:1) in a 50 mL single-necked flask, add 20 mL ethanol to dissolve, ultrasonically shake at 30° C., 40 KHz for 20 min, then add 225.0 mg Na₂Se (molar ratio 1:1:1.8), react under nitrogen protection, 30° C., 40 KHz ultrasonic oscillation. At the end of the reaction, the reaction solution became turbid. The reaction solution was poured into cold water, left standing, and filtered to obtain a crude product, which was then further purified by recrystallization with methanol to obtain compound 2 with a mass of 79.8 mg and a yield of 30.9%.

¹H-NMR (400 MHz, DMSO-d6) δ (ppm): 8.49 (2H, s), 4.17 (2H, m), 2.35 (2H, t), 2.24 (2H, t), 1.92 (2H, m), 1.35 (3H, t); ¹³C-NMR (100 MHz, DMSO-d6) δ (ppm): 165.8, 149.4, 145.6, 141.3, 110.2, 60.2, 44.7, 42.5, 25.2, 12.9.

### Example 3: Preparation of Compound 3

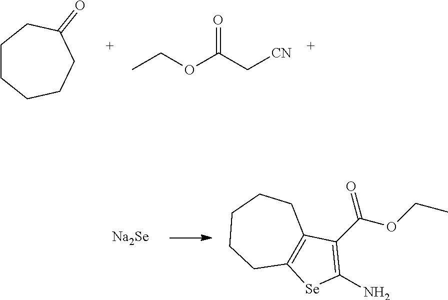

Take 112.0 mg cycloheptanone and 114.0 mg ethyl cyanoacetate (molar ratio 1:1) in a 50 mL single-necked flask, add 20 mL ethanol to dissolve, 60° C., 40 KHz ultrasonic vibration for 20 min, then add 187.5 mg of Na₂Se (molar ratio 1:1:1.5), react under nitrogen protection, 60° C., 40 KHz ultrasonic oscillation. At the end of the reaction, the reaction solution became turbid. The reaction solution was poured into cold water, allowed to stand, and filtered to obtain a crude product, which was then further purified by recrystallization with methanol to obtain compound 3 with a mass of 84.2 mg and a yield of 29.4%.

¹H-NMR (400 MHz, DMSO-d6) δ (ppm): 8.53 (2H, s), 4.29 (2H, m), 2.05 (2H, t), 1.90 (2H, t), 1.62 (2H, m), 1.84 (2H, m), 1.49 (2H, m), 1.40 (3H, t); ¹³C-NMR (100 MHz, DMSO-d6) δ (ppm): 163.1, 151.2, 142.5, 141.8, 110.0, 60.5, 31.8, 31.5, 25.6, 22.3, 15.2.

### Example 4: Preparation of Compound 4

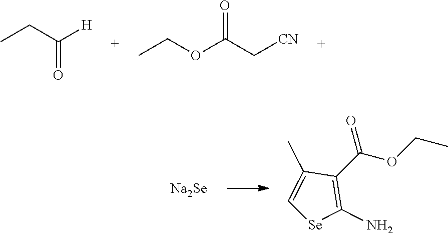

Take 58.0 mg propionaldehyde and 114.0 mg ethyl cyanoacetate (molar ratio 1:1) in a 50 mL single-necked flask, add 20 mL ethanol to dissolve, ultrasonically shake at 30° C., 40 KHz for 20 min, then add 225.0 mg Na₂Se (molar ratio 1:1:1.8), under nitrogen protection, 30° C., 40 KHz ultrasonic oscillation. At the end of the reaction, the reaction solution became turbid. The reaction solution was poured into cold water, allowed to stand, and filtered to obtain a crude product, which was then further purified by recrystallization with methanol to obtain 80.3 mg of compound 4 with a mass of 80.3 mg and a yield of 34.6%.

¹H-NMR (400 MHz, DMSO-d6) δ (ppm): 8.51 (2H, s), 7.36 (1H, s), 4.31 (2H, m), 2.12 (3H, s), 1.36 (3H, t); ¹³C-NMR (100 MHz, DMSO-d6) δ (ppm): 166.7, 145.9, 142.8, 131.9, 113.2, 60.6, 23.0, 13.5.

### Example 5: Preparation of Compound 5

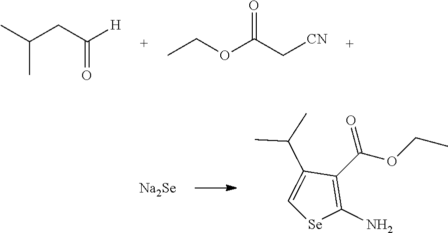

Take 86.0 mg 3-methylbutyraldehyde and 114.0 mg ethyl cyanoacetate (molar ratio 1:1) in a 50 mL single-necked flask, add 20 mL ethanol to dissolve, ultrasonically shake at 30° C., 40 KHz for 20 min, and then add 250 mg Na₂Se (molar ratio 1:1:2), react under nitrogen protection, 30° C., 40 KHz ultrasonic oscillation. At the end of the reaction, the reaction solution became turbid. The reaction solution was poured into cold water, left standing, and filtered to obtain a crude product, which was then further purified by recrystallization with methanol to obtain compound 5 with a mass of 97.3 mg and a yield of 37.4%.

¹H-NMR (400 MHz, DMSO-d6) δ (ppm): 8.47 (2H, s), 7.54 (1H, s), 4.31 (2H, m), 2.60 (1H, m), 1.36 (3H, t), 1.16 (3H, d), 1.11 (3H, d); ¹³C-NMR (100 MHz, DMSO-d6) δ (ppm): 166.2, 155.0, 145.2, 127.9, 110.0, 60.3, 35.2, 22.1, 21.6, 13.5.

### Example 6: Preparation of Compound 6

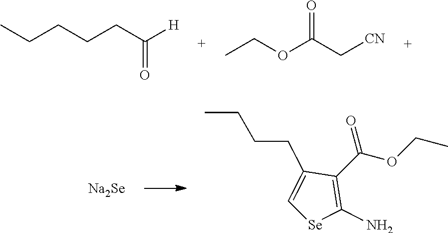

Take 100.0 mg n-hexanal and 114.0 mg ethyl cyanoacetate (molar ratio 1:1) into a 50 mL single-necked flask, add 20 mL ethanol to dissolve, ultrasonically shake at 30° C., 40 KHz for 20 min, then add 250.0 mg Na₂Se (molar ratio 1:1: 2), under nitrogen protection, 30° C., 40 KHz ultrasonic oscillation. At the end of the reaction, the reaction solution became turbid. The reaction solution was poured into cold water, left to stand, and filtered to obtain a crude product, which was then further purified by recrystallization with methanol to obtain compound 6 with a mass of 86.7 mg and a yield of 31.6%.

¹H-NMR (400 MHz, DMSO-d6) 67 (ppm): 8.51 (2H, s), 7.39 (1H, s), 4.09 (2H, m), 2.32 (2H, t), 1.36 (2H, m), 1.25 (3H, t), 1.21 (2H, m), 0.85 (3H, t); ¹³C-NMR (100 MHz, DMSO-d6) δ (ppm): 166.2, 153.6, 145.5, 130.9, 110.0, 63.2, 31.8, 22.6, 15.2, 13.8.

### Example 7: Preparation of Compound 7

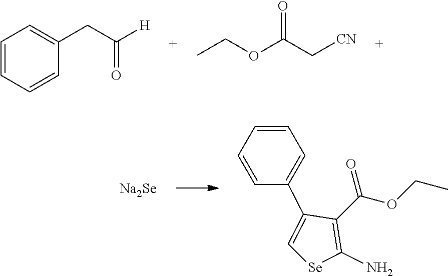

Take 120.0 mg of phenylacetaldehyde and 114.0 mg of ethyl cyanoacetate (molar ratio 1:1) in a 50 mL single-neck flask, add 20 mL ethanol to dissolve, ultrasonically shake at 60° C. and 40 KHz for 20 min, then add 225.0 mg of Na₂Se (molar ratio 1:1:1.8), react under the protection of nitrogen, 60° C., 40 KHz ultrasonic oscillation. At the end of the reaction, the reaction solution became turbid. The reaction solution was poured into cold water, left to stand, and filtered to obtain a crude product, which was then further purified by recrystallization with methanol to obtain compound 7 with a mass of 88.6 mg and a yield of 30.1%.

¹H-NMR (400 MHz, DMSO-d6) δ (ppm): 8.61(2H, s), 8.17 (1H, s), 7.45 (1H, t), 7.42 (1H, t), 7.37 (1H, t), 7.22 (1H, d), 7.19 (1H, d), 4.31 (2H, m), 1.41 (3H, t); ¹³C-NMR (100 MHz, DMSO-d6) δ (ppm): 166.1, 152.1, 142.6, 133.7, 130.1, 130.0, 128.9, 128.9, 117.9, 112.5, 63.6, 13.1.

### Example 8: Preparation of Compound 8

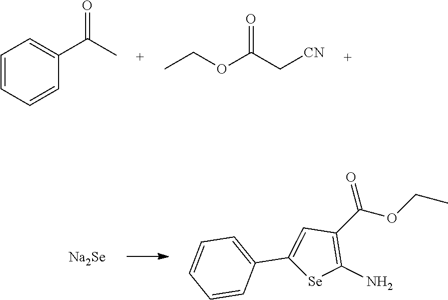

Take 120.0 mg of acetophenone and 114.0 mg of ethyl cyanoacetate (molar ratio 1:1) in a 50 mL single-necked flask, add 20 mL of ethanol to dissolve, ultrasonically shake at 30° C., 40 KHz for 20 min, and then add 187.5 mg of Na₂Se (molar ratio 1:1:1.5), react under nitrogen protection, 30° C., 40 KHz ultrasonic oscillation. At the end of the reaction, the reaction solution became turbid. The reaction solution was poured into cold water, allowed to stand, and filtered to obtain a crude product, which was then further purified by recrystallization with methanol to obtain compound 8 with a mass of 79.7 mg and a yield of 27.1%.

¹H-NMR (400 MHz, DMSO-d6) δ (ppm): 8.52 (2H, s), 7.37 (1H, s), 7.47 (1H, t), 7.45 (1H, t), 7.33 (1H, d), 7.31 (1H, d), 7.25 (1H, t), 4.33 (2H, m), 1.13 (3H, t); ¹³C-NMR (100 MHz, DMSO-d6) δ (ppm): 166.5, 142.7, 137.8, 136.6, 129.9, 129.7, 127.3, 126.5, 126.5, 112.9, 63.6, 12.7.

### Example 9: Preparation of Compound 9

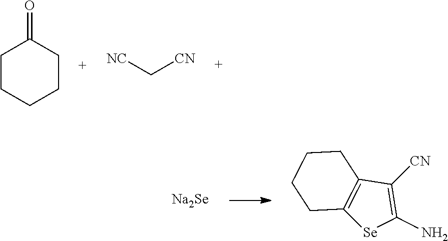 [SMILES: O=C1CCCCC1 ; N#CCC#N ; "Na2Se" ; N#Cc1c(N)[se]c2c1CCCC2]

Take 98.0 mg cyclohexanone and 66.0 mg malononitrile (molar ratio 1:1) in a 50 mL single-necked flask, add 20 mL ethanol to dissolve, ultrasonically shake at 45° C., 40 KHz for 20 min, then add 225.0 mg Na₂Se (molar ratio 1:1:1.8), react under nitrogen protection, 45° C., 40 KHz ultrasonic oscillation. At the end of the reaction, the reaction liquid became turbid, the reaction liquid was poured into cold water, left standing, filtered to obtain a crude product, and then further purified by recrystallization with methanol to obtain compound 9 with a mass of 88.5 mg and a yield of 39.3%.

¹H-NMR (400 MHz, DMSO-d6) δ (ppm): 8.43 (2H, s), 2.47 (2H, t), 1.83 (2H, t), 1.86 (2H, m), 1.63 (2H, m); ¹³C-NMR (100 MHz, DMSO-d6) δ (ppm): 155.1, 150.2, 145.0, 117.0, 93.2, 30.6, 29.8, 28.1, 23.0.

### Example 10: Preparation of Compound 10

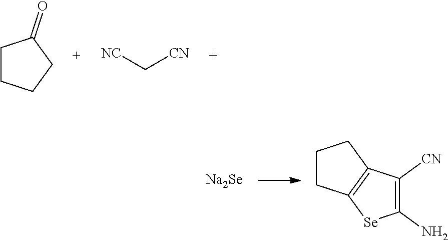 [SMILES: O=C1CCCC1 ; N#CCC#N ; "Na2Se" ; N#Cc1c(N)[se]c2c1CCC2]

Take 84.0 mg of cyclopentanone and 66.0 mg of malononitrile (molar ratio 1:1) in a 50 mL single-necked flask, add 20 mL of ethanol to dissolve, ultrasonically shake at 60° C., 40 KHz for 20 min, then add 225.0 mg of Na₂Se (molar ratio 1:1:2), react under the protection of nitrogen, 60° C., 40 KHz ultrasonic oscillation. At the end of the reaction, the reaction solution became turbid. The reaction solution was poured into cold water, allowed to stand, and filtered to obtain a crude product, which was then further purified by recrystallization with methanol to obtain 65.9 mg of compound with a yield of 31.2%.

¹H-NMR (400 MHz, DMSO-d6) δ (ppm): 8.50 (2H, s), 2.39 (2H, t), 2.17 (2H, t), 1.73 (2H, m); ¹³C-NMR (100 MHz, DMSO-d6) δ (ppm): 152.6, 149.9, 142.1, 113.9, 92.6, 45.6, 42.3, 21.3.

### Example 1: Preparation of Compound 11

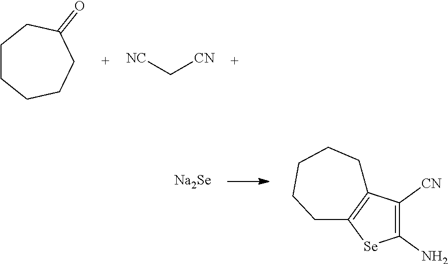 [SMILES: O=C1CCCCCC1 ; N#CCC#N ; "Na2Se" ; N#Cc1c(N)[se]c2c1CCCCC2]

Take 112.0 mg cycloheptanone and 66.0 mg malononitrile (molar ratio 1:1) in a 50 mL single-necked flask, add 20 mL ethanol to dissolve, ultrasonically shake at 45° C., 40 KHz for 20 min, then add 250.0 mg Na₂Se (molar ratio 1:1:2), React under nitrogen protection, 45° C., 40 KHz ultrasonic oscillation. At the end of the reaction, the reaction liquid became turbid, the reaction liquid was poured into cold water, allowed to stand, and filtered to obtain a crude product, which was then further purified by recrystallization with methanol to obtain 71.8 mg of compound with a yield of 30.0%.

¹H-NMR 400 MHz, DMSO-d6) δ (ppm): 8.48 (2H, s), 2.07 (2H, t), 1.83 (2H, t), 1.85 (2H, m), 1.69 (2H, m), 1.51(2H, t); ¹³C-NMR (100 MHz, DMSO-d6) δ (ppm): 155.2, 150.1, 144.8, 114.6, 93.2, 33.6, 32.9, 29.8, 27.9, 23.0.

### Example 12: Preparation of Compound 12

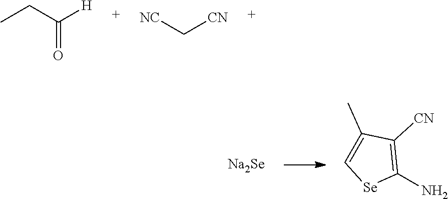

Take 58.0 mg of propionaldehyde and 66.0 mg of malononitrile (molar ratio 1:1) in a 50 mL single-necked flask, add 20 mL of ethanol to dissolve, ultrasonically shake at 30° C., 40 KHz for 20 min, then add 225.0 mg of Na₂Se (molar ratio 1:1:1.8), Under the protection of nitrogen, 30° C., 40 KHz ultrasonic vibration reaction. At the end of the reaction, the reaction liquid became turbid. The reaction liquid was poured into cold water, left standing, and filtered to obtain a crude product, which was then further purified by recrystallization with methanol to obtain 66.6 mg of compound with a yield of 36.0%.

¹H-NMR (400 MHz, DMSO-d6) δ (ppm): 8.63 (2H, s), 7.56 (1H, s), 2.30 (3H, s); ¹³C-NMR (100 MHz, DMSO-d6) δ (ppm): 153.4, 145.9, 134.8, 117.4, 92.5, 20.1.

### Example 1: Preparation of Compound 13

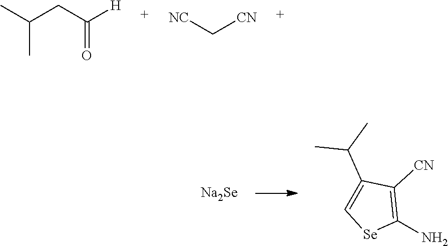

Take 86.0 mg of 3-methylbutyraldehyde and 66.0 mg of malononitrile (molar ratio 1:1) in a 50 mL single-necked flask, add 20 mL ethanol to dissolve, ultrasonically shake at 45° C. and 40 KHz for 20 min, then add 225.0 mg of Na₂Se (molar ratio 1:1:1.8), react under nitrogen protection, 45° C., 40 KHz ultrasonic oscillation. At the end of the reaction, the reaction liquid became turbid, the reaction liquid was poured into cold water, allowed to stand, and filtered to obtain a crude product, which was then further purified by recrystallization with methanol to obtain 82.7 mg of the compound, with a yield of 38.8%.

¹H-NMR (400 MHz, DMSO-d6) δ (ppm): 8.47 (2H, s), 7.58 (1H, s), 2.61 (1H, m), 1.15 (3H, d), 1.14 (3H, d); ¹³C-NMR (100 MHz, DMSO-d6) δ (ppm): 155.1, 152.9, 127.8, 117.3, 93.4, 34.9, 23.2, 23.4.

### Example 14: Preparation of Compound 14

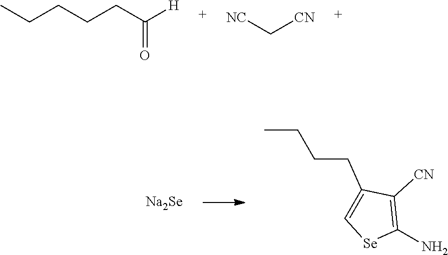

Take 100.0 mg n-hexanal and 66.0 mg malononitrile (molar ratio 1:1) in a 50 mL single-necked flask, add 20 mL ethanol to dissolve, ultrasonically shake at 30° C., 40 KHz for 20 min, then add 187.5 mg of Na₂Se (molar ratio 1:1:1.5), Under nitrogen protection, 30° C., 40 KHz ultrasonic oscillation. At the end of the reaction, the reaction liquid became turbid, the reaction liquid was poured into cold water, left standing, filtered to obtain a crude product, and then further purified by recrystallization with methanol to obtain compound 14 with a mass of 73.6 mg and a yield of 32.4%.

¹H-NMR (400 MHz, DMSO-d6) δ (ppm): 8.44 (2H, s), 7.32 (1H, s), 2.52 (2H, t), 1.44 (2H, m), 1.37 (2H, m), 0.81 (3H, t); ¹³C-NMR (400 MHz, DMSO-d6) δ (ppm): 153.5, 150.1, 134.4, 113.9, 93.4, 31.9, 29.8, 24.9, 15.

### Example 15: Preparation of Compound 15

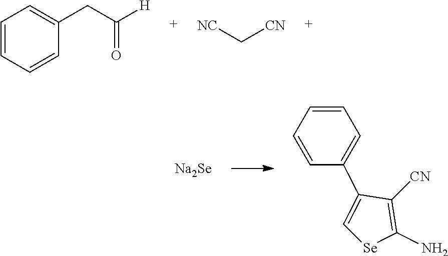 [SMILES: O=CCc1ccccc1 ; N#CCC#N ; "Na2Se" ; N#Cc1c(-c2ccccc2)c[se]c1N]

Take 120.0 mg of phenylacetaldehyde and 66.0 mg of malononitrile (molar ratio 1:1) in a 50 mL single-necked flask, add 20 mL of ethanol to dissolve, 60° C., 40 KHz ultrasonic vibration for 20 min, and then add 187.5 mg of Na₂Se (molar ratio 1:1:1.5), under nitrogen protection, 60° C., 40 KHz ultrasonic oscillation. At the end of the reaction, the reaction solution became turbid, the reaction solution was poured into cold water, left standing, filtered to obtain a crude product, and then further purified by recrystallization with methanol to obtain compound 15 with a mass of 76.4 mg and a yield of 30.9%.

¹H-NMR (400 MHz, DMSO-d6) δ (ppm): 8.65 (2H, s), 8.14 (1H, s), 7.51 (1H, t), 7.50 (1H, t), 7.39 (1H, t), 7.28 (1H, d), 7.27 (1H, d); ¹³C-NMR (100 MHz, DMSO-d6) δ (ppm): 153.2, 151.0, 134.6, 130.2, 130.1, 129.7, 129.6, 121.4, 117.2, 89.7.

### Example 16: Preparation of Compound 16

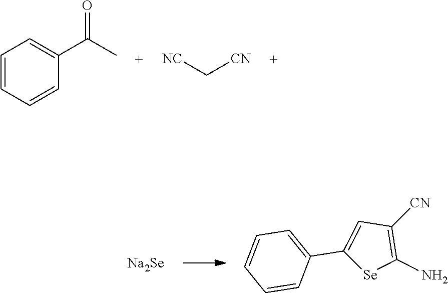

Take 120.0 mg of acetophenone and 66.0 mg of malononitrile (molar ratio 1:1) in a 50 mL single-necked flask, add 20 mL of ethanol to dissolve, ultrasonically shake at 60° C., 40 KHz for 20 min, then add 225.0 mg of Na₂Se (molar ratio 1:1:1.8), React under the protection of nitrogen, 60° C., 40 KHz ultrasonic oscillation. At the end of the reaction, the reaction solution became turbid. The reaction solution was poured into cold water, left to stand, and filtered to obtain a crude product, which was then further purified by recrystallization with methanol to obtain compound 16 with a mass of 71.2 mg and a yield of 28.8%.

¹H-NMR (400 MHz, DMSO-d6) δ (ppm): 8.44 (2H, s), 7.60 (1H, s), 7.56 (1H, t), 7.55 (1H, t), 7.47 (1H, d), 7.45 (1H, d), 7.40 (1H, t); ¹³C-NMR (100 MHz, DMSO-d6) δ (ppm): 153.6, 138.4, 136.5, 130.2, 130.0, 130.0, 129.3, 129.3, 129.2, 117.5, 93.3.

### Example 17: Compound's In Vitro Antibacterial Activity Determination

Using micro broth dilution method, with ceftazidime and vancomycin as positive controls, test the minimum inhibitory concentration (MIC) of compounds 9, 11, and 14.

The experimental strains included drug-resistant gram-positive bacteria: methicillin-resistant Staphylococcus aureus MRSA 18-575, 18-596; drug-resistant gram-negative bacteria: multi-drug-resistant Pseudomonas aeruginosa MDR-PA 18-126, 18-756. The experimental strains were all donated by Huashan Hospital Affiliated to Fudan University (Fudan University Antibiotic Research Institute), and used after being identified by conventional methods.

The specific steps are as follows:

Preparation of MHB medium: Weigh 20.0 g of MHB medium, add it to 1L of distilled water, heat and boil until it is completely dissolved, divide it into conical flasks, autoclave at 121° C. for 15 minutes, and set aside.

Cultivate the experimental strains to the logarithmic growth phase: under aseptic conditions, inoculate the experimental strains into 100 mL of MHB medium, and place them in a constant temperature and humidity incubator at 37° C. for 20-22 hours for use.

Preparation of stock solution: Weigh the sample to be tested and dissolve it with 1% DMSO solution to prepare a stock solution with a concentration of 2560 μg/mL; weigh the positive control substance and dissolve it with sterile distilled water to prepare a stock solution with a concentration of 2560 μg/mL.

Bacterial suspension preparation: Under aseptic conditions, the experimental strains cultured to the logarithmic growth phase are calibrated to 0.5 McDonald's unit turbidity standard with MHB medium and diluted at a ratio of 1:10 to prepare a concentration of 10⁶ CFU/mL Bacterial suspension, spare.

Stock solution dilution and inoculation of experimental strains: under aseptic conditions, dilute the stock solution to a solution of 256 μg/mL. Take a sterile 96-well plate, add 100 μL of MHB medium to each well except for wells 1-3; add 100 μL of positive control solution to well 2 and 100 μL of compound sample solution to wells 3 and 4; Mix the sample solution in the well with the culture medium, and then pipet 100 μL to the fifth well. After mixing, pipet 100 μL to the sixth well, and then dilute to the tenth well by successive times, and then take 100 μL from the tenth well and discard it. The 11th well is a growth control without drugs; then, add 100 μL of the above-prepared bacterial suspension to each well, so that the final bacterial concentration in each well is 5×10⁵ CFU/mL; at this point, the positive control concentration is 128 μg/mL, the concentration of the sample solution is 128, 64, 32, 16, 8, 4, 2, 1 μg/mL.

Incubation: Cover the 96-well plate that has been inoculated with experimental strains and place it in a constant temperature and humidity box at 37° C. for 20-22 hours.

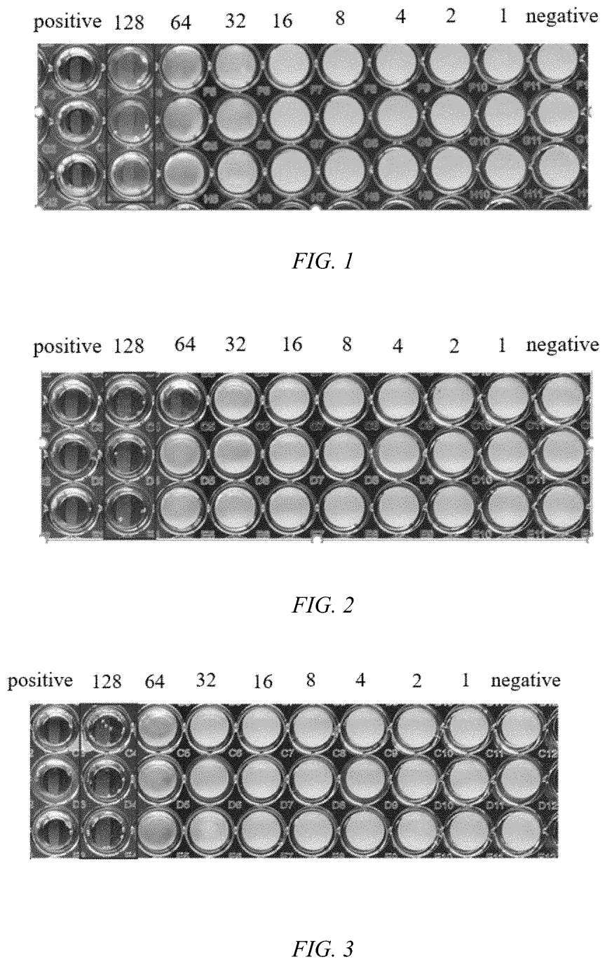

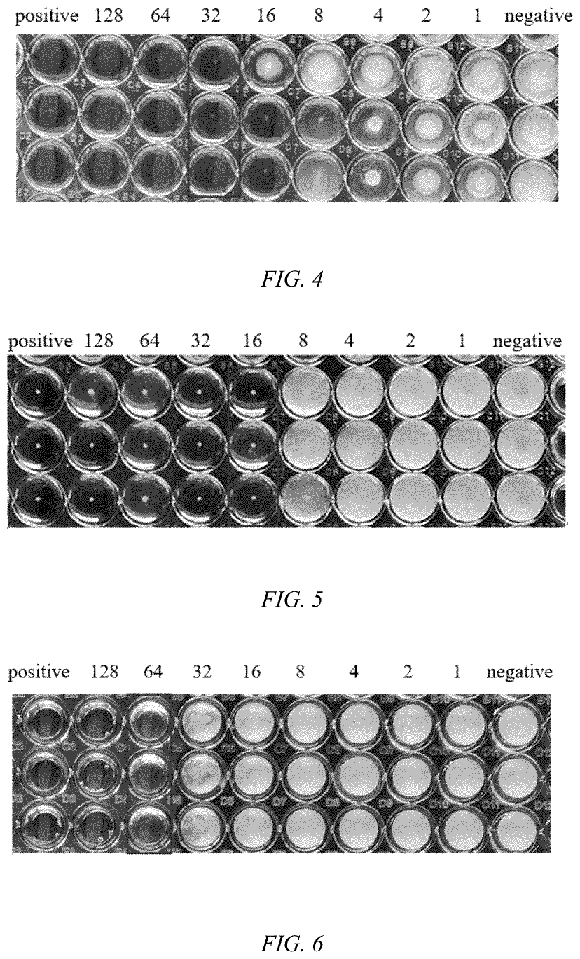

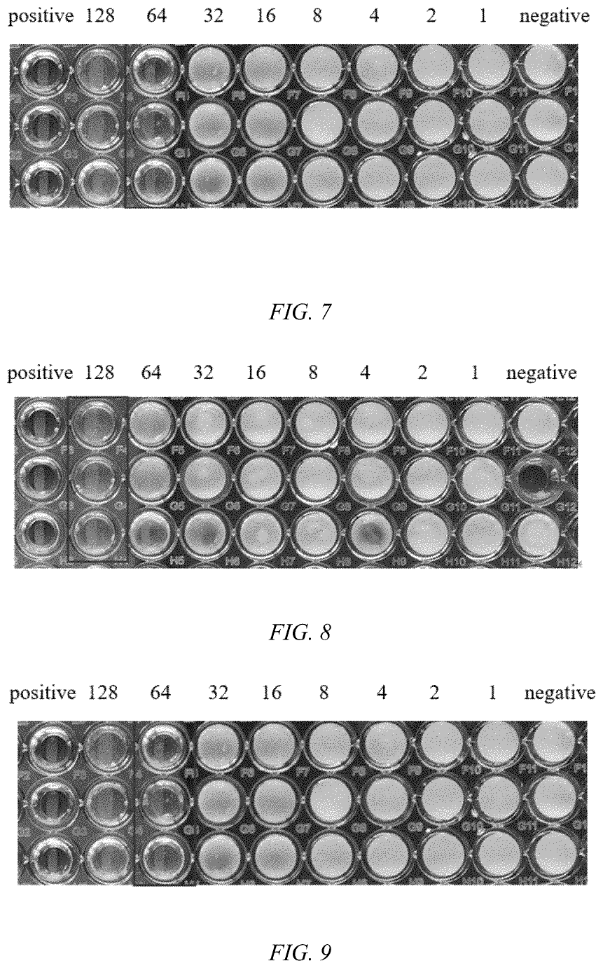

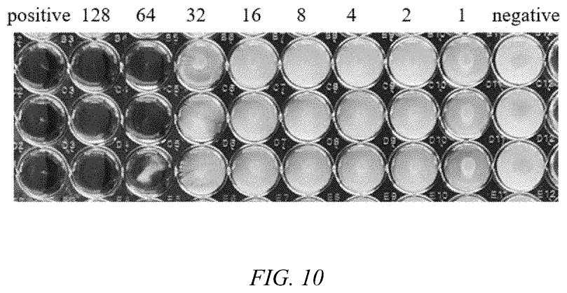

MIC endpoint interpretation: The concentration that can completely inhibit the growth of bacteria seen in a 96-well plate under a black background is the minimum inhibitory concentration of the sample for the bacteria. Record the results as shown in FIGS. 1 to 10 (the holes are in order from left to right corresponding to positive, 128, 64, 32, 16, 8, 4, 2, 1, negative) and Table 1.

TABLE 1
The minimum inhibitory concentration of tested drugs
and positive drugs (μg · mL⁻¹)

| Strain | |
|---|---|
| MRSA | MDR-PA |

| Sample | 18-575 | 18-596 | 18-126 | 18-756 |
|---|---|---|---|---|
| Compound 9 | 128 | 128 | >128 | 128 |
| Compound 11 | 32 | 16 | 64 | 64 |
| Compound 14 | 128 | 64 | 64 | >128 |
| Ceftazidime | 128 | 128 | 128 | 128 |
| Vancomycin | 512 | 512 | \ | \ |

The results of Table 1 and FIGS. 1-10 show that compounds 9, 11, and 14 have anti-multidrug resistance bacteria activity, among which compound 11 is resistant to drug-resistant gram-positive bacteria MRSA (MIC=16 μg/mL) and drug-resistant gram The positive bacteria MDR-PA (MIC=64 μg/mL) showed a stronger inhibitory effect, and it was stronger than the positive control drug. In summary, the compounds 9, 11, and 14 of the present invention can be used as antibacterial drug candidates for methicillin-resistant Staphylococcus aureus and multi-drug resistant Pseudomonas aeruginosa, and further preclinical researches can be carried out.

What is claimed is:

1. A compound with anti-drug resistant bacteria activity having the following formula (I): 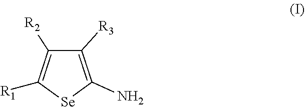
wherein,
R¹ and R² are each independently H, alkyl, or phenyl, or R₁ and R₂ form —(CH₂)₃—, —(CH₂)₄—, or —(CH₂)₅—, and
R₃ is —COOCH₂CH₃, or CN.

2. The compound of claim 1, wherein the compound is selected from the group consisting of:

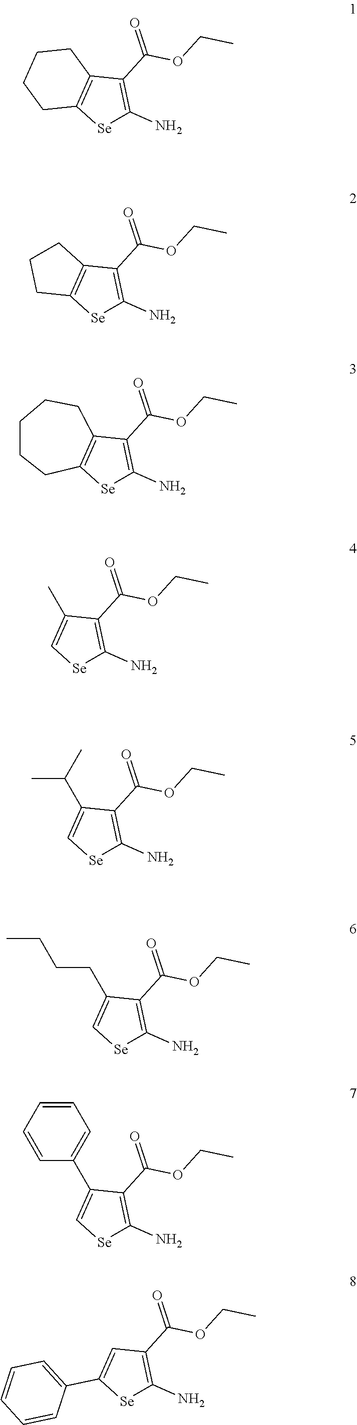

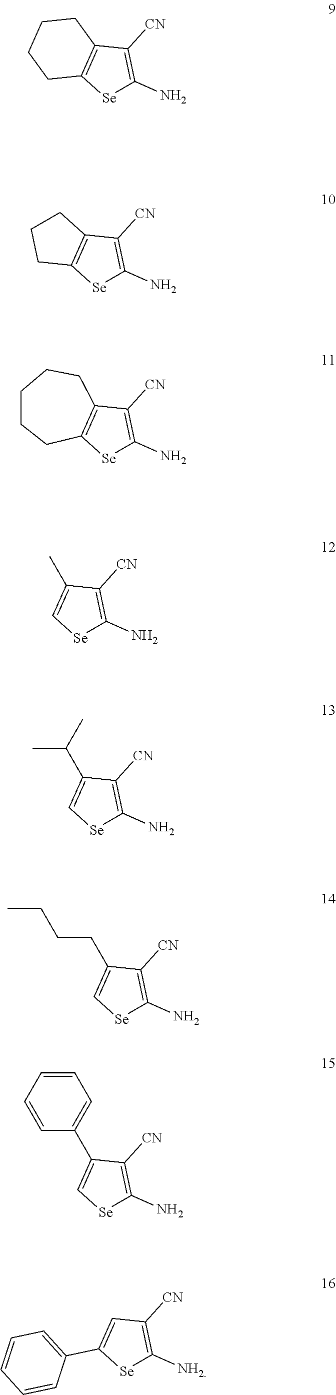

3. A method of preparing the compound of formula (I) of claim 1, comprising:
reacting a compound of formula (II) with a compound of formula (III) and Na₂Se to obtain the compound of formula (I):

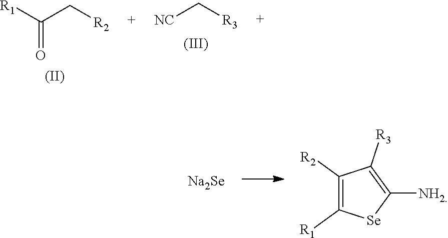

4. The method of claim 3, wherein the method comprises the following steps:
placing the compound of formula (II), the compound of formula (III) and an organic solvent in a reactor;
adding Na₂Se under nitrogen atmosphere to obtain a reaction mixture;
heating the reaction mixture at 30-60° C. for 2-5 hours;
pouring the reaction mixture system into ice water, standing still for crystallization to obtain a crude product; and
recrystallizing the crude product using methanol and drying to obtain the compound of formula (I).

5. The method of claim 4, wherein the organic solvent is methanol or ethanol.

6. The method of claim 4, wherein a molar ratio of the compound of formula (II), the compound of formula (III) and Na₂Se is 1:1:(1.5-2).

7. The method of claim 6, wherein the molar ratio of the compound of formula (II), the compound of formula (III) and Na2Se is 1:1:1.8.

8. The method of claim 4, wherein the reaction mixture is heated at 45° C.

9. The method of claim 4, wherein the reaction mixture is heated for 3 hours.
